(19) United States
(12) Patent Application Publication
(10) Pub. No.: US 2014/0121373 A1
(43) Pub. Date: May 1, 2014

(54) PROCESS FOR PREPARING STABLE POLYMORPHIC FORM OF ERLOTINIB HYDROCHLORIDE

(71) Applicants: Nikhil Amar Singh, Gujarat (IN); Gulab Khushalrao Pathe, Gujarat (IN); Dinesh Kumar Chaurasiya, Gujarat (IN); Kumar Kamlesh Laxmi Singh, Gujarat (IN)

(72) Inventors: Nikhil Amar Singh, Gujarat (IN); Gulab Khushalrao Pathe, Gujarat (IN); Dinesh Kumar Chaurasiya, Gujarat (IN); Kumar Kamlesh Laxmi Singh, Gujarat (IN)

(73) Assignee: CADILA HEALTHCARE LIMITED, Ahmedabad (IN)

(21) Appl. No.: 14/115,253

(22) PCT Filed: Apr. 23, 2012
(86) PCT No.: PCT/IN2012/000295

§ 371 Date: Dec. 3, 2013

(30) Foreign Application Priority Data
May 3, 2011 (IN) 1371/MUM/2011

(51) Int. Cl. C07D 239/94

(52) U.S. Cl. CPC C07D 239/94
USPC 544/293

(57) ABSTRACT

The present invention discloses an improved and efficient process for preparing Erlotinib hydrochloride suitable as a cancer drug.

### FIELD OF INVENTION

The present invention discloses an improved and efficient process for preparing a stable polymorphic form of Erlotinib hydrochloride used as an anti cancer drug.

### BACKGROUND OF THE INVENTION

Erlotinib is chemically described as 4-(3-Ethynylphenylamino)-6,7-bis(2-methoxyethoxy)-quinazoline and sold as Tarceva (R) which is the hydrochloride salt of Erlotinib; it has the following formula (I).

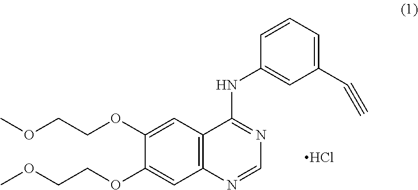 [SMILES: C#Cc1cccc(Nc2ncnc3cc(OCCOC)c(OCCOC)cc23)c1 ; Cl]

Erlotinib is a once-a-day, orally active inhibitor of the epidermal growth factor receptor (EGFR) tyrosine kinase. This small molecule is one of a class of anticancer drugs that target the underlying molecular mechanisms involving oncogenes and tumor suppressor genes that play critical role in the conversion of normal cells into a cancerous state. Erlotinib specifically targets the epidermal growth factor receptor (EGFR) tyrosine kinase, which is highly expressed and occasionally mutated in various forms of cancer.

Polymorphism is defined as “the ability of a substance to exist as two or more crystalline phases that have different arrangement and/or conformations of the molecules in the crystal Lattice. Thus, in the strict sense, polymorphs are different crystalline forms of the same pure substance in which the molecules have different arrangements and/or different configurations of the molecules”. Different polymorphs may differ in their physical properties such as melting point, solubility, X-ray diffraction patterns, etc. Polymorphic forms of a compound can be distinguished in the laboratory by analytical methods such as X-ray diffraction (XRD), Differential Scanning Calorimetry (DSC) and Infrared spectrometry (IR).

Solvent medium and mode of crystallization play very important role in obtaining a crystalline from over the other.

Erlotinib hydrochloride can exist in different polymorphic forms, which differ from each other in term of stability, physical properties, spectral data and methods of preparation.

U.S. Pat. No. 5,747,498 discloses process for preparation of Erlotinib and its hydrochloride salt, which is useful for the treatment of cancer. The general process for the preparation is shown in below scheme:

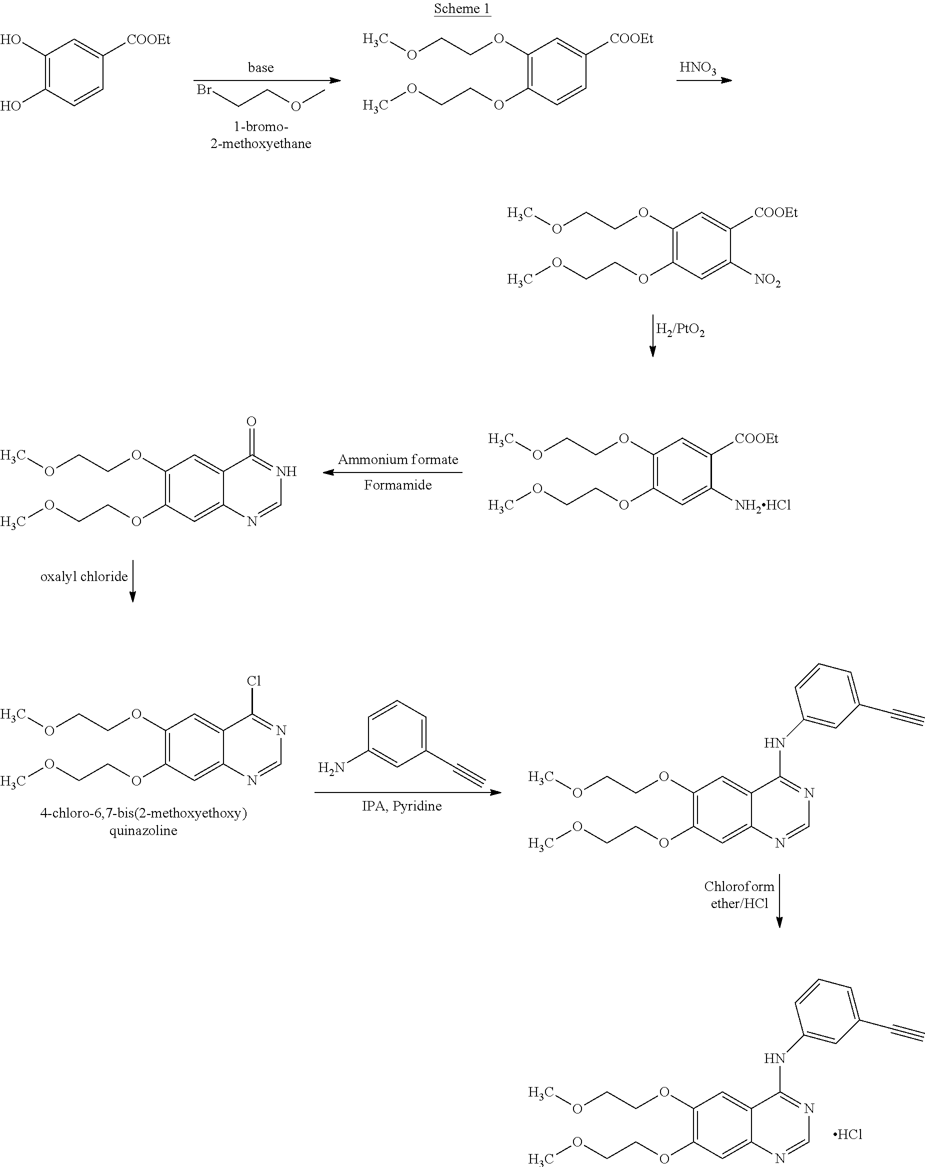

The process involves the condensation of 4-chloro-6,7-bis(2-methoxyethoxy)quinazoline with 3-ethynylaniline in presence of pyridine in isopropanol to prepare Erlotinib, which is purified by using flash chromatography and is subsequently converted to its hydrochloride salt.

U.S. Pat. No. 6,900,221 discloses the preparation of polymorph B from either polymorph A or a mixture of polymorph A and B of Erlotinib hydrochloride. The process involves hydrolysis of 4-(3-aminophenyl)-2-methyl-but-3-yn-2-ol to 3-ethynyl aniline in presence of sodium hydroxide in toluene. The solution of 3-ethynyl aniline in toluene is coupled with 4-chloro-6,7-bis(2-methoxyethoxy)quinazoline in acetonitrile to give Erlotinib hydrochloride from A or mixture of forms A and B.

U.S. Pat. No. 7,148,231 discloses a novel polymorph E, and a process for preparation of the polymorphic form E of Erlotinib hydrochloride. The process involves the coupling between 4-chloro-6,7-bis(2-methoxyethoxy)quinazoline, with 3-ethynylaniline in presence of α,α,α-triflouro toluene and HCl.

WO 2007138613 disclosed novel processes for the preparation of Erlotinib hydrochloride. The process involves reaction of 3,4-dihydroxy benzaldehyde with bromo derivative of ethyl methyl ether to obtain 3,4-bis-(2-methoxyethoxy)benzaldehyde. This is converted to give 3,4-bis-(2-methoxyethoxy)-benzonitrile, which on further nitration gives 4,5-bis(2-methoxyethoxy)-2-nitrobenzonitrile. The compound 4,5-bis(2-methoxyethoxy)-2-nitrobenzonitrile is subsequently reduced to give 2-amino-4,5-bis(2-methoxyethoxy)benzonitrile. Formylation with derivative of formic acid of 2-amino-4,5-bis(2-methoxyethoxy)benzonitrile yields N′-[2-cyano-4,5-bis(2methoxyethoxy)phenyl]-N,N-dimethylfromamidine. Coupling of this N′-[2-cyano-4,5-bis(2methoxyethoxy)phenyl]-N,N-dimethylfromamidine with 3-ethynyl aniline gives Erlotinib free base. Further treatment of this free base with methanolic/ethanolic hydrochloric acid gives Erlotinib hydrochloride.

WO 2007138612 disclosed another process for the preparation of Erlotinib hydrochloride. The process involves reacting 3,4-dihydroxy benzaldehyde with bromo derivative of ethyl methyl ether in an inert solvent to obtain 3,4-bis(2-methoxyethoxy)benzaldehyde which is further converted into 3,4-bis(2-methoxyethoxy)-benzonitrile in the presence of base. The nitration of compound 3,4-bis(2-methoxyethoxy)-benzonitrile further gives 4,5-bis(2-methoxyethoxy)-2-nitrobenzonitrile which on reduction gives 2-amino-4,5-bis(2-methoxyethoxy)benzonitrile which is subsequently reacted with N′-(3-ethynylphenyl)-N,N-dimethyl fromamidine to give Erlotinib free base. Further treatment of this free base with methanolic/ethanolic hydrochloric acid gives Erlotinib hydrochloride

WO 2008102369 discloses process and preparation for Form-M, Form-N and Form-P of Erlotinib hydrochloride each of which is characterized by characteristic two θ values.

X-ray Powder Diffraction Pattern for Form-M, Form-N and Form-P are:
(i) Form-M: having typical characteristic peaks at about 6.2, 7.9, 9.6, 11.4, 12.5, 13.4, 14.7, 15.7, 17.0, 17.6, 19.2, 20.2, 20.7, 21.1, 21.9, 22.4, 23.0, 23.9, 24.4, 25.1, 25.9, 26.8, 29.0, 29.7, 31.7, 32.7, 34.8, 40.2 on the 2θ value.
(ii) Form-N: having typical characteristic peaks at about 5.56, 9.72, 11.25, 12.82, 18.84, 19.38, 21.01, 22.74, 23.46, 24.23, 25.34, 26.70, 29.17, 32.77, 37.21, 39.96, 45.66 on the 2θ value.
(iii) Form-P: having typical characteristic peaks at about 2.97, 5.80, 6.36, 9.97, 10.54, 11.48, 15.00, 15.80, 16.64, 17.11, 17.62, 18.15, 18.58, 19.06, 19.78, 20.74, 22.14, 22.96, 23.72, 24.45, 25.67, 26.40, 27.30, 28.14, 28.76, 29.44, 30.15, 30.82, 32.21, 32.95, 33.99, 34.59, 40.49, 40.64, 42.02, 43.87 on the 2θ value.

U.S. Patent Application Publication No 2009131665 discloses a process for the preparation of crystalline Erlotinib hydrochloride. The process includes the coupling of 4-chloro-6,7-bis(2-methoxyethoxy)quinazoline with 3-ethynyl aniline in isopropyl alcohol as solvent to give Erlotinib hydrochloride.

U.S. Patent Application Publication No. 20090012295 discloses polymorphs G1, G2, G3, as well as amorphous form of Erlotinib base, and processes for the preparation thereof.

WO 2010005924 discloses a process for preparing crystalline form of Erlotinib base form G2 characterized by data selected from the group consisting of: an X-ray powder diffraction pattern with peaks at about 6.5, 12.9, 17.3, 18.3 and 22.4 degrees two-θ±0.2 degrees two-θ. The process comprises reacting sodium acetate and

Erlotinib hydrochloride in an alcohol to obtain a precipitate containing crystalline Erlotinib base form G2.

Thus, the known process involves very expensive technique like flash chromatography for the purification of the Erlotinib base, which is not commercially viable. Therefore, there is a need to develop a process, which overcomes one or more of the above limitations. We hereinafter describe a process which is commercially viable, economical and removes the drawback of prior art.

### Embodiments of the Invention

The objective of present invention is to provide an improved process for the preparation of stable polymorphic form of Erlotinib hydrochloride. The process comprises the steps of:
1) chlorinating the compound of formula IV with suitable chlorinating reagent in the presence of suitable solvent to get compound of formula III. Optionally, suitable anti-solvent may be used.

Preferably, the chlorinating reagent is selected from thionyl chloride, phosphoryl chloride, phosphorous pentachloride, oxalyl chloride, methane sulphonyl chloride, benzene sulphonyl chloride, aniline sulphonyl chloride.

Preferably, the suitable solvent selected for chlorination is selected from toluene, dichloromethane, chloroform, acetonitrile, cyclohexane and the like or mixture thereof.

Preferably, the suitable anti-solvent is selected from isopropyl alcohol, heptane, hexane, toluene and the like or mixture thereof.
2) reacting compound of formula III with 3-ethynyl benzenamine (3-EBA) in the presence of suitable base and suitable solvent to get compound of formula II.

Preferably, the suitable base is selected from pyridine, triethyl amine, n-methyl morpholine, collidine, 2-6-lutidine, N, N dimethyl aniline and the like.

Preferably, the suitable solvent is selected from C₁ to C₆ alcohols such as methanol, ethanol, propanol, isopropanol, butanol, pentanol, hexanol and the like; chlorinated solvents selected from dichloromethane, chloroform and the like; DMF; N-methylpyrrolidin-2-one; acetonitrile; tetrahydrofuran; 1,4-dioxane or mixture thereof.
3) optionally, purification of compound of formula H with suitable solvent to get the pure compound of formula II. Optionally, suitable anti-solvent may be added.

Preferably, the suitable solvent is selected from chloroform, methanol, butan-2-one, ethyl acetate, water, acetone and the like or mixture thereof.

Preferably, the suitable anti-solvents are selected from toluene, hexane, methanol, water, ether and the like or mixture thereof.
4) the purified compound of formula II is further reacted with concentrated HCl or solution of HCl in suitable solvent to give a stable polymorphic form of Erlotinib hydrochloride of formula I.

Preferably, the Erlotinib free base of compound of formula II is dissolved in suitable solvent(s) selected from chloroform, methyl isobutyl ketone, isopropyl acetate, acetone, acetonitrile, dichloromethane, dioxane, ether and like or mixture thereof.

Preferably, the HCl is first added to solution. The solution can be an organic solution such as ether, isopropyl alcohol or aqueous solution.

According to another objective of invention is to provide process for the purification of crude Erlotinib base of formula II with suitable solvent, optionally in the presence of suitable anti-solvent to get pure Erlotinib base of formula II.

Preferably, the suitable solvent is selected from chloroform, methanol, butan-2-one, ethyl acetate, water, acetone and the like or mixture thereof. Preferably, the suitable anti-solvent selected from toluene, hexane, water, ether and the like or mixture thereof.

In another embodiment is provided a stable polymorphic form of Erlotinib hydrochloride characterized by Differential Scanning Calorimetry (DSC) having one sharp endotherm peak in the range between 214-224° C. and another endotherm sharp peak in the range between 229-239° C.

In another embodiment, the stable polymorphic form of Erlotinib hydrochloride is further characterized by its melting point in the range or 229±3° C.

In yet another embodiment, the stable polymorphic form of Erlotinib hydrochloride of the present invention is still further characterized by PXRD peaks at 2θ of 5.5, 18.8, 22.7, 24.6 and 26.1±0.2 degrees.

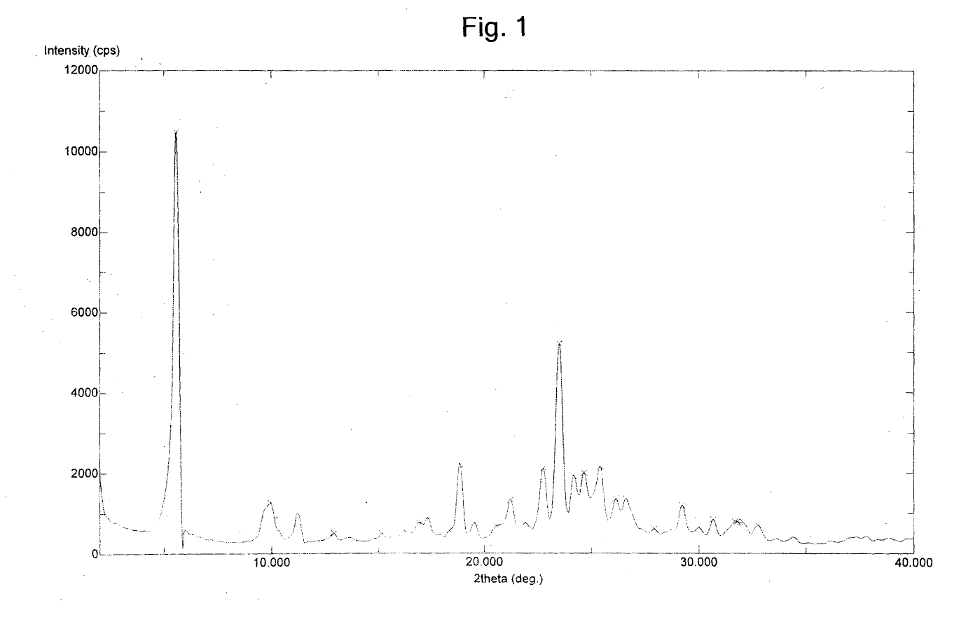

In a still further embodiment, the stable polymorphic form of Erlotinib hydrochloride of the present invention is still further characterized by a PXRD pattern as provided in FIG. 1.

According to another object of the present invention is to provide a pharmaceutical composition comprising stable polymorphic form of Erlotinib hydrochloride with suitable pharmaceutical excipients and carriers.

### BRIEF DESCRIPTION OF THE INVENTION

FIG. 1 illustrates the powder X-ray diffraction pattern of stable polymorphic form of Erlotinib hydrochloride.

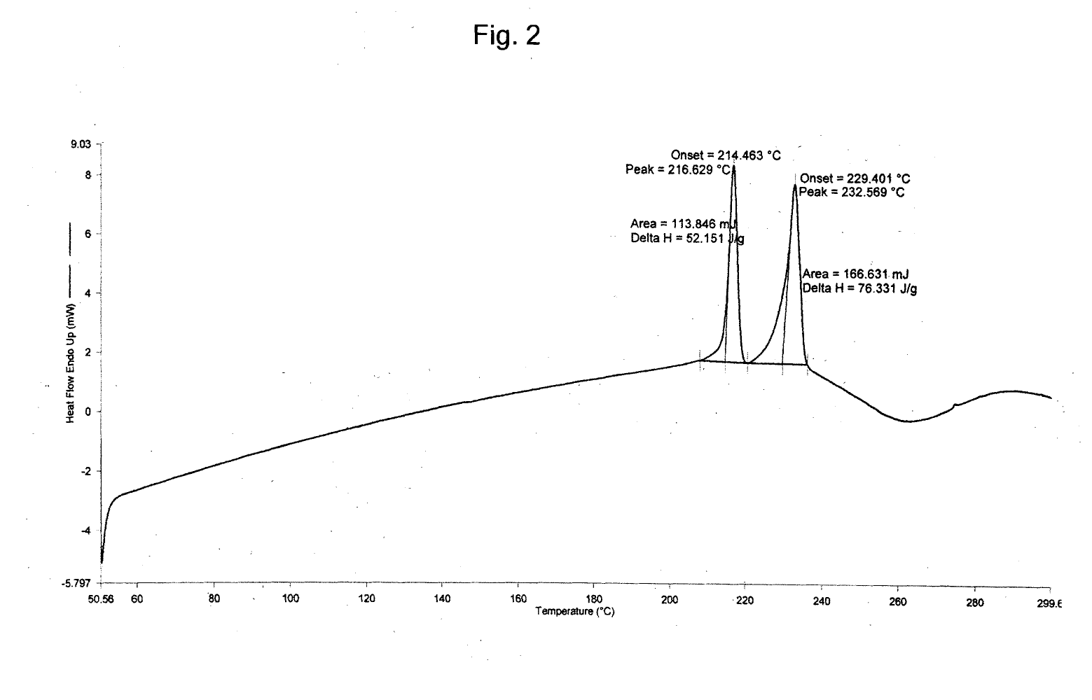

FIG. 2 illustrates the DSC of stable polymorphic form of Erlotinib hydrochloride.

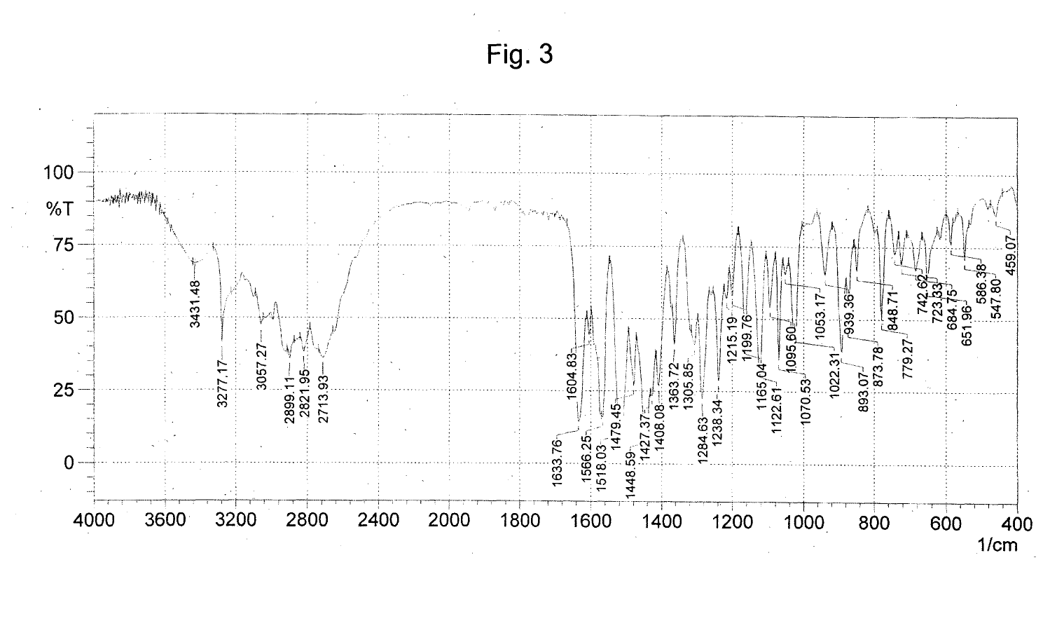

FIG. 3 illustrates the IR pattern of stable polymorphic form of Erlotinib hydrochloride.

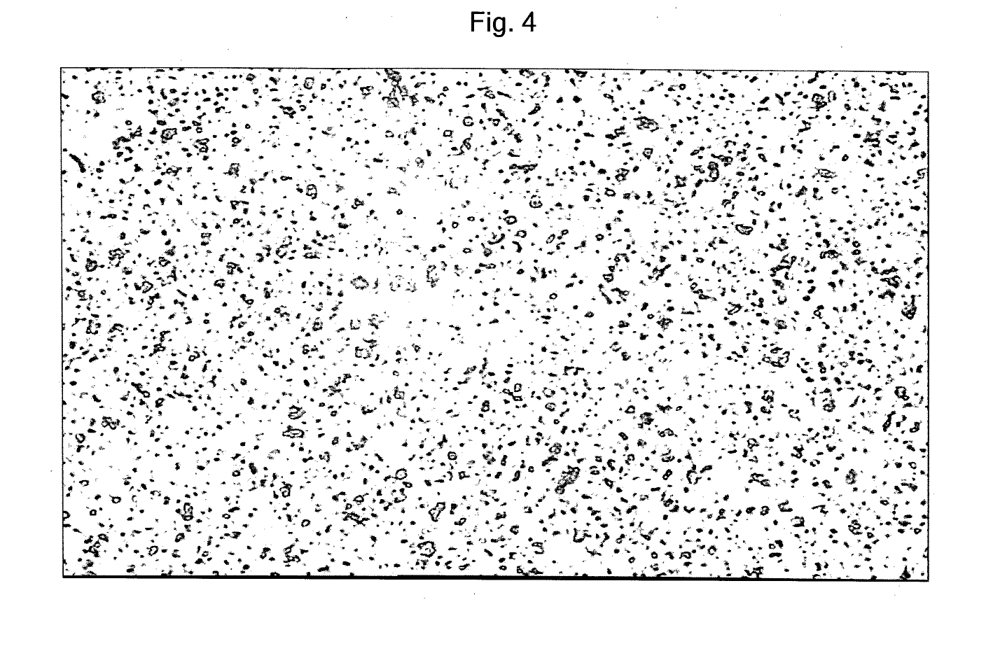

FIG. 4 illustrates the Microscopic view of stable polymorphic form of Erlotinib hydrochloride.

### DETAILED DESCRIPTION OF THE INVENTION:

As used herein, the term “THF” refers to tetrahydrofuran, the term “DCM” refers to dichloromethane, the term “DMF” refers to dimethyl formamide, the term “DIPE” refers to diisopropyl ether, the term “DMSO” refers to dimethyl sulphoxide, the term BMEQ is 6,7-bis(2-methoxyethoxy)quinazolin-4(3H)-one, the term (4-chloro-6,7-bis(2-methoxyethoxy)quinazoline) is herein after referred to as ClBMEQ and the term “RM” refers the reaction mass, the term “RT” refers to room temperature, (25-30° C.) “MTBE” refers to Methyl tertiary butyl ether, “MDC” refers to dichloromethane, “3-EBA” refers to 3-ethynyl benzenamine.

The present invention provides an improved process for the preparation of Erlotinib hydrochloride as depicted in Scheme 2. The present invention also provides for a stable polymorphic form of Erlotinib hydrochloride. The stable form of Erlotinib hydrochloride as per the present invention is characterized by
A DSC having one sharp endotherm peak in the range between 214-224° C. and another endotherm sharp peak in the range between 229-239° C. as provided in FIG. 2;
A melting point of 229±3° C.;
A PXRD pattern having characteristic PXRD peaks at 2θ of 5.5, 18.8, 22.7, 24.6 and 26.1±0.2 degrees
A PXRD pattern substantially as depicted in FIG. 1 of the specification;
An IR pattern as provided in FIG. 3 of the specification;
A microscopic pattern as provided in FIG. 4 of the specification.

The process for the preparation of Erlotinib hydrochloride of the present invention is as described in Scheme 2.

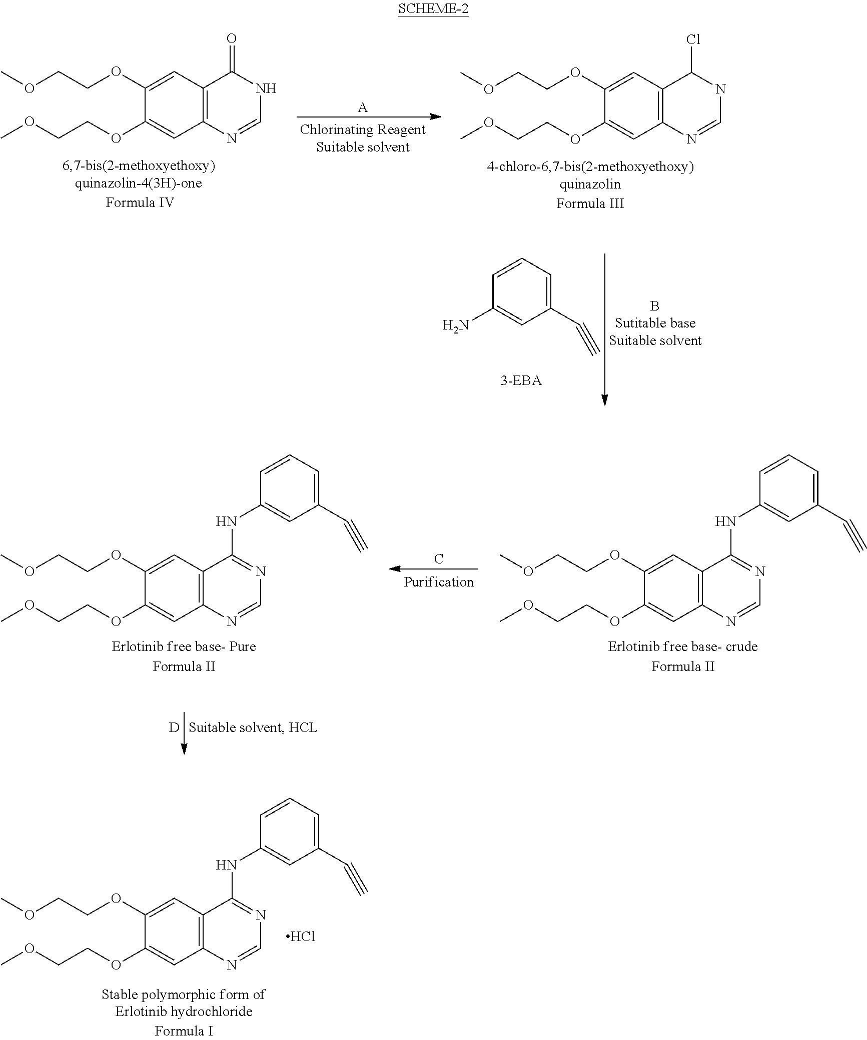

Accordingly, the invention provides a process for the preparation of stable polymorphic form of Erlotinib hydrochloride comprising the steps of:
Step A: Chlorinating the compound of formula IV (6,7-bis(2-methoxyethoxy)quinazolin-4(3H)-one) with suitable chlorinating reagent in the presence of suitable solvent and optionally suitable anti-solvent to get compound of formula III (4-chloro-6,7-bis(2-methoxyethoxy)quinazoline).

Preferably, the chlorinating reagent selected from thionyl chloride, phosphoryl chloride, phosphorous pentachloride, oxalyl chloride, methane sulphonyl chloride, benzene sulphonyl chloride, aniline, sulphonyl chloride, either used alone or in combination.

Preferably, the suitable solvent selected for chlorination is selected from toluene, dichloromethane, chloroform, acetonitrile, cyclohexane and the like or mixture thereof.

Preferably, the suitable anti-solvent is selected from isopropyl alcohol, heptane, hexane, toluene and the like or mixture thereof.
Step B: Reacting the compound of formula III (4-chloro-6,7-bis(2-methoxyethoxy)quinazoline) with 3-ethynyl benzenamine (3-EBA) in the presence of suitable base and suitable solvent to get compound of formula II (crude Erlotinib free base).

Preferably, the suitable base selected from pyridine, triethyl amine, n-methyl morpholine, collidine, 2-6-lutidine, N, N dimethyl aniline and the like either used alone or in suitable combination.

Preferably, the suitable solvent is selected from C₁ to C₆ alcohols such as methanol, ethanol, propanol, isopropanol, butanol, isobutanol, pentanol, hexanol and the like, chlorinated solvents selected from dichloromethane, chloroform and the like, DMF, N-methylpyrrolidin-2-one, acetonitrile, tetrahydrofuran, 1,4-dioxane or suitable mixtures thereof.
Step C: Optionally, purification of compound of formula II with suitable solvent and optionally in the presence of, suitable anti-solvent to get pure Erlotinib free base of formula II.

Preferably, the suitable solvent is selected from chloroform, methanol, butan-2-one, ethyl acetate, water, acetone and the like or mixture thereof

Preferably, the suitable anti-solvent is selected from toluene, hexane, water, ether and the like or mixture thereof.
Step D: The purified compound of formula II is further reacted with concentrated HCl or solution of HCl in suitable solvent to give stable polymorphic form of Erlotinib hydrochloride of formula I.

Preferably, the Erlotinib free base of compound of formula II is dissolved in suitable solvent selected from chloroform, Methyl isobutyl ketone, isopropyl acetate, acetone, acetonitrile, dichloromethane, dioxane, ether and like or mixture thereof

Preferably, the HCl is first added to solvent. The solvent can be an organic solvent such as suitable ethers, alcohols such as isopropyl alcohol or an aqueous solution.

In an another embodiments of invention is to provide a process for purification of crude Erlotinib base of formula II with suitable solvent and optionally in the presence of suitable anti-solvent to get pure Erlotinib base of formula II.

Preferably, the suitable solvent is selected from chloroform, methanol, butan-2-one, ethyl acetate, water, acetone and the like or mixture thereof.

Preferably, the suitable anti-solvent is selected from toluene, hexane, water, ether and the like or mixture thereof.

An important feature of the present invention is that the Erlotinib hydrochloride obtained by the disclosed process has a high stability which is further confirmed by stability data as provided below:

Chemical Stability Data of the stable form of Erlotinib hydrochloride

| | | The Erlotinib hydrochloride packed in double HMHDPE bag as a primary packing and this bag is | | | | | | | | |
|---|---|---|---|---|---|---|---|---|---|---|
| | | further protected with triple laminated aluminum bag kept in fiber drum. | | | | | | | | |
| | | | | Imp-1 | Imp-2 | Imp-3 | AOSUI | Total | m/c | Assay (%), |
| Packing | | | Description | (%) | (%) | (%) | (%) | Imp | (%) | OAB |
| Procedure | | | LIMIT | | | | | | | |
| S | Storage | Months | White to | | | | | | | |
| No | Condition | (1-6) | off white | 0.15 w/w | 0.15 w/w | 0.15 w/w | 0.1 w/w | 0.1 w/w | 0.5 w/w | 98.0-102 w/w |
| 1 | 5° C. ± 3° C. | Month 1 | Complies | 0.05 | 0.07 | ND | ND | 0.12 | 0.16 | 99.5 |
| | (Refrigerator) | Month 2 | Complies | 0.05 | 0.08 | ND | ND | 0.13 | 0.15 | 99.6 |
| | | Month 3 | Complies | 0.05 | 0.08 | ND | ND | 0.13 | 0.15 | 99.5 |
| | | Month 6 | Complies | 0.06 | 0.08 | ND | ND | 0.14 | 0.15 | 99.8 |
| 2 | 25° C. ± 2° C./ | Month 1 | Complies | 0.05 | 0.08 | ND | ND | 0.13 | 0.14 | 99.9 |
| | 60% ± 5% | Month 2 | Complies | 0.05 | 0.08 | ND | ND | 0.13 | 0.13 | 99.2 |
| | RH | Month 3 | Complies | 0.05 | 0.08 | ND | ND | 0.13 | 0.14 | 99.3 |
| | | Month 6 | Complies | 0.05 | 0.08 | ND | ND | 0.13 | 0.14 | 99.9 |
| 3 | 30° C. ± 2° C./ | Month 1 | Complies | 0.06 | 0.08 | ND | ND | 0.14 | 0.12 | 99.6 |
| | 65% ± 5% | Month 2 | Complies | 0.05 | 0.08 | ND | ND | 0.13 | 0.15 | 99.4 |
| | RH (Real | Month 3 | Complies | 0.05 | 0.08 | ND | ND | 0.13 | 0.15 | 99.4 |
| | Time) | Month 6 | Complies | 0.06 | 0.08 | ND | ND | 0.14 | 0.14 | 99.8 |
| 4 | 40° C. ± 2° C./ | Month 1 | Complies | 0.06 | 0.08 | ND | ND | 0.14 | 0.15 | 99.3 |
| | 75% ± 5% | Month 2 | Complies | 0.05 | 0.08 | ND | ND | 0.13 | 0.16 | 99.5 |
| | RH | Month 3 | Complies | 0.05 | 0.08 | ND | ND | 0.13 | 0.16 | 99.9 |
| | | Month 6 | Complies | 0.06 | 0.08 | ND | ND | 0.14 | 0.17 | 99.4 |

*The abbreviation used in above table is defined as follows:
Imp. 1 (Impurity-1) = N-(3-Ethynylphenyl)-6-(2-chloroethoxy)-7-(2-methoxyethoxy)-quinazolin-4-amine hydrochloride
Imp. 2 (Impurity-2) = N-(3-Ethynylphenyl)-7-(2-chloroethoxy)-6-(2-methoxyethoxy)-quinazolin-4-amine hydrochloride
Imp. 3 (Impurity-3) = N-(3-Ethynylphenyl)-6,7-bis-(2-chloroethoxy) quinazolin-4-amine Hydrochloride
AOSUI = Any other specified unknown impurity

The above table shows the chemical stability for six months period at different condition such as 5° C.±3° C. (Refrigerator), 25° C.±2° C.±5% RH, 30° C.±2° C./65%±5% RH (Real Time) and 40° C.±2° C./75%±5% RH of Erlotinib Hydrochloride

This study ensures that the Erlotinib Hydrochloride according to the invention is stable at least upto 6 months with all impurities in limit.

Polymorphic Stability Data of the stable form
of Erlotinib Hydrochloride
Packing Procedure The Erlotinib hydrochloride packed
in double HMHDPE bag as a primary packing
and this bag is further protected with triple
laminated aluminum bag kept in fiber drum.

| | | | PXRD | DSC | IR |
|---|---|---|---|---|---|
| | | | Should comply with data of stable | | |
| | Storage | | polymorphic from of | | |
| S No | Conditions | Months (1-6) | Erlotinib hydrochloride | | |
| 1 | 5° C. ± 3° C. | Month 1 | Complies | Complies | Complies |
| | (Refrigerator) | Month 2 | Complies | Complies | Complies |
| | | Month 3 | Complies | Complies | Complies |
| | | Month 6 | Complies | Complies | Complies |
| 2 | 25° C. ± 2° C./ | Month 1 | Complies | Complies | Complies |
| | 60% ±5% RH | Month 2 | Complies | Complies | Complies |
| | | Month 3 | Complies | Complies | Complies |
| | | Month 6 | Complies | Complies | Complies |
| 3 | 30° C. ± 2° C./ | Month 1 | Complies | Complies | Complies |
| | 65% ±5% RH | Month 2 | Complies | Complies | Complies |
| | (Real Time) | Month 3 | Complies | Complies | Complies |
| | | Month 6 | Complies | Complies | Complies |
| 4 | 40° C. ± 2° C./ | Month 1 | Complies | Complies | Complies |
| | 75% ±5% RH | Month 2 | Complies | Complies | Complies |
| | | Month 3 | Complies | Complies | Complies |
| | | Month 6 | Complies | Complies | Complies |

The above table shows the polymorphic stability for six months period at different condition such as 5° C.±3° C. (Refrigerator), 25° C.±2° C./60%±5% RH, 30° C.±2° C./65%±5% RH (Real Time) and 40° C.±2° C./75%±5% RH of the stable form of Erlotinib Hydrochloride.

The above two studies ensures that the Erlotinib Hydrochloride according to present invention retained the same polymorphic and chemical identity at least up to six months.

The water content of the batch of Erlotinib Hydrochloride as per the present invention was found to be the same and does not vary as provided below:
Initial water content: 0.14% w/w
Water content after 3 months: 0.14% w/w
Water content after 6 months: 0.14% w/w

The HPLC single maximum impurity content of the batch of Erlotinib Hydrochloride prepared according to the present invention was as follows:
Initial single maximum impurity content: 0.08% w/w
Single maximum impurity content after 3 months: 0.08% w/w
Single maximum impurity content after 6 months: 0.08% w/w

The HPLC total impurity content of the batch of the stable form of Erlotinib Hydrochloride was found not to vary as provided below:
Initial total impurity content: 0.13% w/w
Total impurity content after 3 months: 0.13% w/w
Total impurity content after 6 months: 0.13% w/w

The above data shows that Erlotinib hydrochloride obtained as per the present disclosed process is stable under different conditions. Therefore, the Erlotinib hydrochloride prepared by the process of the present application is substantially pure both chemically and in terms of polymorphic content and is highly stable due to which it is suitable for preparation of pharmaceutical composition which can be used even in hot environments such as in Asia and Africa.

According to another aspect of the present invention is provided a pharmaceutical composition comprising the stable polymorphic form of Erlotinib hydrochloride of the present invention with suitable pharmaceutical excipients and carriers. Such a formulation will be useful in tropical countries also having very high temperatures.

The present application provides an improved process for preparing Erlotinib hydrochloride, having the following advantages:

1. High yield

2. High Purity

3. Removal of costly technique like Flash Chromatography.

4. Additionally, the process also yields a highly stable Polymorph.

5. Commercially viable process.

The stable polymorphic form of Erlotinib hydrochloride of the present invention may be used to obtain the Form A as well as Form B of Erlotinib hydrochloride in pure and stable form.

The process is further described by the following non-limiting examples, which provides the preferred mode of carrying out the process of the present invention. It is to be appreciated that several alterations, modifications, optimizations of the processes described herein are well within the scope of a person skilled in the art and such alterations, modifications, optimizations etc. should be construed to be within the scope of the present inventive concept as is disclosed anywhere in the specification. Further the examples should not be construed as limiting the scope of the invention in any way.

### Instrument Analysis:

Powder X-ray Diffraction: X-ray powder diffraction spectrum was observed on a MF 2100 2 KW X-ray Powder diffractometer of make Rigaku having a Copper Kα-radiation at a voltage of 40 kV and 30 mA. Approximately 150 mg sample was gently flattened on a quartz plate without further processing (e.g. Grinding and sieving) and scanned from 4° to 40° at 0.010° sampling width and 4.000° per minute.
IR Spectroscopy: The IR spectra was performed on 8400S of make Shimadzu by using KBr pellet and recorded from 4000 to 400 cm⁻¹. About 2 mg of sample was triturated with 300 mg of finely powdered and dried potassium bromide. The mixture was carefully grinded, spread uniformly in a suitable die and submitted it in vacuum to a pressure of about 800 MPa (80 kg/cm²). From the FTIR spectrum a blank FT-IR spectrum of KBr was subtracted. The blank IR spectrum was recorded prior to the measurement of the samples.

Differential Scanning Calorimetry (DSC): Melting points are determined by means of a DSC thermogram using a Pyris-1 make Perkin Elmer. DSC (Differential Scanning Calorimetry) is the technique of dynamic differential Calorimetry. Using this technique, the melting temperature can be measured by heating the sample until a thermal, i.e. an endothermic or exothermic, reaction is detected by means of ultrasensitive sensors. The DSC cell/sample chamber was purged with 100 ml/min of ultra-high purity nitrogen. The instrument was calibrated with high purity Indium. The sample was placed into an open aluminum DSC pan and measured against an empty reference pan. About 2 mg of sample being placed into the bottom of the pan and lightly tapped down to ensure good contact with the pan. The instrument was programmed to heat at a heating rate of 10° C./min in the temperature range between 50° C. and 300° C.
Microscope: A Lieca Microscope with Linkam Hot stage microscopy (TMS-94) system with polarized light, CCD camera and Linksys 32 data software.
HPLC: High Performance Liquid Chromatography Separation Module with PDA detector, Make Waters, Model No 2695.
KF: Moisture content was analyzed by using Karl Fisher Autotitrator 795 KFT Titrino make Metrohm.

### EXAMPLE 1

### Preparation of 4-Chloro-6, 7-bis-(2-methoxyethoxy)quinazoline (ClBMEQ)

Into a reaction vessel, BMEQ (1 gm, 3.4 mmol) and thionyl chloride (6 ml, 82.71 mmol) was added under stirring and nitrogen purging. Further DMF (0.1 ml, 1.3 mmol) was added in reaction mass and refluxed for 1-4 hours. The excess thionyl chloride was evaporated under vacuum and cooled the residue to room temperature and added 5 ml dichloromethane and 5 ml saturated solution of sodium bicarbonate with continuous stirring. The organic layer was separated and washed with saturated solution of sodium bicarbonate and water. The organic layer was separated, dried over sodium sulphate and concentrated the organic layer till one volume of MDC remains. To this was added 10 ml of Isopropyl alcohol, cooled to 0-5° C., stirred for 30 min. Filtered the material and air dried under 9 mbar vacuum for 30 min to get 856 mg ClBMEQ (Yield=80.8%, Purity=98.70%).

### EXAMPLE 2

### Preparation of 4-Chloro-6,7-bis-(2-methoxyethoxy)quinazoline (ClBMEQ)

Into a reaction vessel, BMEQ (1 gm, 3.4 mmol) and thionyl chloride (6 ml, 82.71 mmol) was added under stirring and nitrogen purging. Further DMF (0.1 ml, 1.3 mmol) was added in reaction mass and reflux for 2 hour. The excess thionyl chloride was evaporated under vacuum, further cooled the residue to room temperature and added 5 ml dichloromethane and 5 ml saturated solution of sodium bicarbonate with continuous stirring. The organic layer was separated and washed with saturated solution of sodium bicarbonate and water. The organic layer was separated, dried over sodium sulphate and concentrated the organic layer till one volume of MDC remains. Then 10 ml of Isopropyl alcohol was added into RM, cooled to 0-5° C., stirred for 30 min. Filtered the material and air dried under 9 mbar vacuum for 30 min to get 927 mg ClBMEQ (Yield=87.5%, Purity=83.79%).

### Example 3

### Preparation of 4-Chloro-6,7-bis-(2-methoxyethoxy)quinazoline (ClBMEQ)

Into a reaction vessel, BMEQ (1 gm, 3.4 mmol) and 10 ml MDC was added under stirring and nitrogen purging. Then DMF (0.12 ml, 1.61 mmol) was added in RM and cool to 20-30° C. The Thionyl chloride (1.65 ml, 22.75 mmol) was added dropwise while maintaining the temperature between 20-30° C. Reflux the RM up to 5 hour. The pH of RM was maintained 6 to 7 by using 10% NaOH solution. The organic layer was separated, dried over sodium sulfate and concentrated the organic layer. 10 ml of n-Heptane was added to the residue and stirred. The RM was cooled to 10-15° C., stirred for 1 hour, filtered, washed with n-Heptane and air dried the compound under 9 mbar vacuum for 30 min to get 929 mg ClBMEQ (Yield=87.6%, Purity=97.65%).

### EXAMPLE 4

### Preparation of 4-Chloro-6,7-bis-(2-methoxyethoxy)quinazoline (ClBMEQ)

Into a reaction vessel, BMEQ (1 gm, 3.4 mmol) and Thionyl chloride (6 ml, 82.71 mmol) was added under stirring and nitrogen purging. Further DMF (0.1 ml, 1.3 mmol) was added in reaction mass and refluxed for 2 hour. The excess thionyl chloride was evaporated under vacuum and cooled the residue to room temperature and added 4 ml dichloromethane and 4 ml saturated solution of sodium bicarbonate with continuous stirring. The organic layer was separated and washed with water. The organic layer was separated, dried over sodium sulphate and concentrated the organic layer till one volume of MDC remains. Then 10 ml of Isopropyl alcohol was added in RM, cooled to 0-5° C., stirred for 30 min. Filtered the material and air dried under 9 mbar vacuum for 30 min to get 643 mg ClBMEQ (Yield=60.7%, Purity=95.36%).

### Example 5

### Preparation of 4-Chloro-6,7-bis-(2-methoxyethoxy)quinazoline (ClBMEQ)

Into a reaction vessel, BMEQ (1 gm, 3.4 mmol) and pyridine (0.56 ml, 6.95 mmol) was added under stirring and nitrogen purging. The RM was cooled to 0-5° C., added phosphoryl chloride (4.07 ml, 43.66 mmol) dropwise and made the temp to RT. The RM was maintained at reflux for 2.5 hour. The RM was concentrated under vacuum at 60° C. and 28 ml chloroform was added in residue which was further added into 18 ml saturated cooled (10-15° C.) solution of sodium bicarbonate with stirring. Stirred for 10 min, separated the layers, washed with brine solution and concentrated the organic layer at 50° C. under 9 mbar vacuum till dryness to get 1.03 gm ClBMEQ (Yield=97.2%, Purity=82.41%).

### EXAMPLE 6

### Preparation of 4-Chloro-6,7-bis-(2-methoxyethoxy)quinazoline (ClBMEQ)

Into a reaction vessel, BMEQ (1 gm, 3.4 mmol) and 10 ml chloroform was added under stirring and nitrogen purging. Dimethyl formamide (0.12 ml, 1.6 mmol) was added in RM and cooled to 5-10° C. Further thionyl chloride (1.65 ml, 22.75 mmol) was added dropwise at 5-10° C. The RM was further reflux for 5 hours and cooled to RT and made the pH of RM to 7 to 8 by using 10% NaOH solution. The organic layer was separated, dried over sodium sulfate and concentrated the organic layer till two volume remains. Further 20 ml n-Heptane was added in RM and cooled to 0-5° C., stirred for 1 H, filtered, washed with n-Heptane and air dried the compound under 9 mbar vacuum for 30 min to get 820 mg ClBMEQ (Yield=77.4%, Purity=96.21%).

### EXAMPLE 7

### Preparation of 4-Chloro-6,7-bis-(2-methoxyethoxy)quinazoline (ClBMEQ)

Into a reaction vessel, BMEQ (1 gm, 3.4 mmol) and 10 ml MDC was added under stirring and nitrogen purging. DMF (0.25 ml, 3.4 mmol) was added in RM and cool to 5-10° C. Further thionyl chloride (0.617 ml, 8.5 mmol) was added dropwise at 5-10° C. and made the temp to RT. The RM was further reflux for 5 hour, then cooled the RM to 5-10° C. and added 5 ml cold water and made the pH of RM to 7.5 to 8 by using 20% NaOH solution. The organic layer, was separated washed with 10 ml brine solution, dried over sodium sulfate and concentrated the organic layer till two vol of MDC remains. Further 20 ml n-Heptane was added in RM and cooled to 0-5° C., stirred for 1 H, filtered, washed with n-Heptane and dried the compound at 40° C. under 9 mbar Vacuum for 4 hour to get 878 mg ClBMEQ (Yield=82.8%, Purity=98.80%).

### EXAMPLE 8

### Preparation of 4-Chloro-6,7-bis-(2-methoxvethoxyv)quinazoline (ClBMEQ)

Into a reaction vessel, BMEQ (1 gm, 3.4 mmol) and 10 ml MDC was added under stirring and nitrogen purging. DMF (0.125 ml, 1.7 mmol) was added in RM and cool to 5-10° C. Further thionyl chloride (0.5 ml, 6.9 mmol) was added dropwise at 5-10° C., made the temp to RT. The RM was maintained at reflux for 5 hour then cooled to RT further to 5-10° C. and added 5 ml cold water and made the pH of RM to 7.5 to 8 by using 20% NaOH solution. The organic layer, was separated washed with brine solution, dried over sodium sulfate and concentrated the organic layer till two volume of MDC remains. Further 20 ml of toluene was added in RM and cooled to 0-5° C., stirred for 1 H, filtered, washed with toluene and dried the compound at 40° C. under 2 mbar vacuum for 4 hours to get 880 mg ClBMEQ (Yield=83.0%, Purity=85.27%).

### EXAMPLE 9

### Preparation of 4-Chloro-6,7-bis-(2-methoxyethoxy)quinazoline (ClBMEQ)

Into a reaction vessel, BMEQ (1 gm, 3.4 mmol) and 10 ml MDC was added under stirring and nitrogen purging. DMF (0.125 ml, 1.7 mmol) was added in RM and cool to 5-10° C. Further thionyl chloride (0.617 ml, 8.5 mmol) was added dropwise at 5-10° C. and made the temp to RT. The RM was maintained at reflux for 5 hour then cooled to RT further to 5-10° C. and added 5 ml cold water and made the pH of RM to 7.5 to 8 by using 20% NaOH solution. The organic layer was separated, dried over sodium sulfate, concentrated the organic layer and added 15 ml n-hexane in RM and cooled to 0-5° C., stirred for 1 H, filtered, washed with n-hexane and dried the compound at 50° C. under 9 mbar vacuum for 4 hour to get 903 mg ClBMEQ (Yield=85.2%, Purity=95.43%).

### EXAMPLE 10

### Preparation of Erlotinib Free Base (Crude)

Into a reaction vessel, added 17 ml IPA, pyridine (0.28 ml, 3.5 mmol), 3-EBA (0.75 gm, 6.4 mmol) and ClBMEQ (1 gm, 3.2 mmol) under stirring and nitrogen purging. The RM was maintained at reflux for 4 hour. The solvent was evaporated under vacuum at 50° C. and added 20 ml solution of chloroform: methanol (9:1) and 20 ml saturated solution of sodium bicarbonate in the solid compound under stirring. Stirred the RM for 10 min. The organic layer was separated, dried over sodium sulphate and distilled off the solvent under 2 mbar vacuum at 50° C. till dry to get 760 mg Erlotinib free base (crude) (Yield=60.4%, Purity=88.84%).

### EXAMPLE 11

### Preparation of Erlotinib Free Base (Crude)

Into a reaction vessel, 17 ml IPA, Pyridine (0.28 ml, 3.52 mmol), 3-EBA (0.936 gm, 7.99 mmol) and ClBMEQ (1 gm, 3.20 mmol) was added under stirring and nitrogen purging. The RM was maintained at reflux for 7 hour. The solvent was evaporated under vacuum at 50° C. and added 20 ml solution of chloroform: methanol (9:1) and 20 ml saturated solution of sodium bicarbonate in the above residue under stirring. Stirred the RM for 10 min. The organic layer was separated, dried over sodium sulphate and distilled off the solvent under 9 mbar Vacuum at 50° C. to get 1.245 gm Erlotinib free base (crude) (Yield=98.9%, Purity=89.84%).

### Example 12

### Preparation of Erlotinib Free Base (Crude)

Into a reaction vessel, added 34 ml IPA, pyridine (0.56 ml, 6.99 mmol), 3-EBA (1.124 gm, 9.6 mmol) and ClBMEQ (2 gm, 6.4 mmol) under stirring and nitrogen purging. The RM was maintained at reflux for 4 hour. The RM was cooled to RT and filtered the solid compound then washed with IPA and added 20 ml solution of chloroform: methanol (9:1) and 20 ml saturated solution of sodium bicarbonate in the solid compound under stirring. Stirred the RM for 10 min. The organic layer was separated, dried over sodium sulphate and distilled off the solvent under vacuum at 50° C. till dryness to get 2.049 gm Erlotinib free base (crude) (Yield=81.5%, Purity=98.92%).

### EXAMPLE 13

### Preparation of Erlotinib Free Base (Crude)

Into a reaction vessel, added 85 ml IPA, pyridine (1.41 ml, 17.49 mmol), S-EBA (2.05 gm, 17.49 mmol) and C1BMEQ (5 gm, 15.98 mmol) under stirring and nitrogen purging The RM was maintained at reflux 4 hour. The RM was cooled to RT and filtered the solid compound then washed with IPA and added 50 ml solution of chloroform: methanol (9:1) and 50 ml saturated solution of sodium bicarbonate in the solid compound under stirring. Stirred the RM for 15 min. The organic layer was separated, dried over sodium sulphate and distilled off the solvent under vacuum at 50° C. till dryness to get 5.22 gm Erlotinib free base (crude) (Yield=83.0%, Purity=98.69%).

### EXAMPLE 14

### Preparation of Erlotinib Free Base (Crude)

Into a reaction vessel, 34 ml IPA, Pyridine (0.56 ml, 7 mmol), 3-EBA (0.82 gm, 7 mmol) and ClBMEQ (2 gm, 6.40 mmol) in a reaction vessel under stirring and nitrogen purging. The RM was maintained at reflux 4 hour. The RM was cooled to RT and filtered the solid compound then washed with IPA and added 20 ml solution of chloroform:methanol (9:1) and 20 ml saturated solution of sodium bicarbonate in the solid compound under stirring. Stirred the RM for 10 min. The organic layer was separated, dried over sodium sulphate and distilled off the solvent under vacuum at 50° C. till dryness. Into the residue 40 ml toluene was added and stirred for 1 hour at 25-30° C. The RM was filtered, washed with toluene and dried the compound under 2 mbar vacuum at 50° C. for 4 hour to get 1.98 gm Erlotinib free base (crude) (Yield=79.0%, Purity=99.38%).

### EXAMPLE 15

### Preparation of Erlotinib Free Base (Crude)

Into a reaction vessel, added 17 ml IPA, pyridine (029 ml, 3.59 mmol), 3-EBA (0.42 gm, 3.59 mmol) and ClBMEQ (1 gm, 3.2 mmol) under stirring and nitrogen purging. The RM was maintained at reflux for 4 hour. The RM was cooled to RT and filtered the solid compound then washed with IPA and added 10 ml solution of chloroform:methanol (9:1) and 10 ml saturated solution of sodium bicarbonate in the solid compound under stirring. Stirred the RM for 15 min. The organic layer was separated, dried over sodium sulphate and distilled off the solvent under vacuum at 50° C. till 2 volume remains. Into the residue 20 ml toluene was added dropwise and stirred for 1 H at RT. The RM was filtered, washed with 2 ml toluene and air dried the compound under 2 mbar vacuum for 30 min to get 0.83 gm Erlotinib free base (crude) (Yield=65.7%, Purity=99.40%).

### EXAMPLE 16

### Preparation of Erlotinib Free Base (Crude)

Into a reaction vessel, added 17 ml IPA, pyridine (0.387 ml, 4.8 mmol), 3-EBA (0.562 gm, 4.8 mmol) and ClBMEQ (1 gm, 3.2 mmol) under stirring and nitrogen purging. The RM was maintained at reflux for 4 hour. The RM was cooled to RT and filtered the solid compound then washed with IPA and added 10 ml solution of chloroform: methanol (9:1) and 10 ml saturated solution of sodium bicarbonate in the solid compound under stirring. Stirred the RM for 15 min. The organic layer was separated, dried over sodium sulphate and distilled off the solvent under vacuum at 50° C. till 2 volume remains. Into residue 20 ml toluene was added dropwise and stirred for 1 H at 0-5° C. Filtered, washed with 2 ml toluene and air dried the compound under 2 mbar Vacuum for 30 min to get 1.09 gm Erlotinib free base (crude) (Yield=86.7%, Purity=98.31%).

### EXAMPLE 17

### Preparation of Erlotinib Free Base (Crude)

Into a reaction vessel, added 17 ml IPA, Triethyl amine (0.49 ml, 3.52 mmol), 3-EBA (0.41 gm, 3.52 mmol) and ClBMEQ (1 gm, 3.2 mmol) under stirring and nitrogen purging. The RM was maintained at reflux till the reaction not completed. The RM was cooled to RT and filtered the solid compound then washed with IPA and added 10 ml solution of chloroform: methanol (9:1) and 10 ml saturated solution of sodium bicarbonate in the solid compound under stirring. Stirred the RM for 15 min.

The organic layer was separated, dried over sodium sulphate and distilled off the solvent under vacuum at 50° C. till 2 volume remains. Into the residue 20 ml hexane was added dropwise and stirred for 1 H at 0-5° C. Filtered, washed with 2 ml hexane and air dried the compound under 2 mbar vacuum for 30 min to get 0.89 gm Erlotinib free base (crude) (Yield=70.9%, Purity=99.36%).

### EXAMPLE 18

### Preparation of Erlotinib Free Base (Crude)

Into a reaction vessel, added 17 ml IPA, pyridine (0.26 ml, 3.20 mmol), 3-EBA (0.375 gm, 3.20 mmol) and ClBMEQ (1 gm, 3.20 mmol) under stirring and nitrogen purging. The RM was maintained at reflux for 5 hour. The RM was filtered, then washed with IPA and added 25 ml solution of chloroform: methanol (9:1) and 25 ml saturated solution of sodium bicarbonate in the solid compound under stirring. Stirred the RM for 15 min. Separated the organic layer, dried over sodium sulphate and distilled off the solvent under vacuum at 50° C. till dryness to get 0.94 gm Erlotinib free base (crude) (Yield=74.7%, Purity=94.72%).

### EXAMPLE 19

### Preparation of Pure Erlotinib Free Base

Into a reaction vessel charged Erlotinib free base crude (200 mg, 0.51 mmol) and 2 ml acetone in a reaction vessel and heat to 60° C. till clear solution obtains. Added 10 mg charcoal and stirred for 10 min. Filtered the RM on hyflo bed and washed with 0.5 ml acetone. Cooled the filtrate to RT then to 0-5° C. and stirred for 1 H. Filtered and dried at 60° C. under 9 mbar vacuum for 4 H to get 64 mg ERL free base purified (Yield=32.0%, Purity=98.93%).

### EXAMPLE 20

### Preparation of Pure Erlotinib Free Base

Into a reaction vessel charged Erlotinib free base crude (1 gm, 2.54 mmol) and 10 ml acetone in a reaction vessel and heat to 60° C. till clear solution obtains. Added 50 mg charcoal and stirred for 10 min. Filtered the RM on hyflo bed and washed with 2 ml acetone. Cooled the filtrate to RT then to 0-5° C. and stirred for 1 H. Filtered and dried at 60° C. under 9 mbar vacuum for 4 H to get 650 mg ERL free base purified (Yield=65.0%, Purity=99.01%).

### EXAMPLE 21

### Preparation of Pure Erlotinib Free Base

Into a reaction vessel charged Erlotinib free base crude (1.5 gm, 3.81 mmol) and 15 ml acetone in a reaction vessel and heat to 45° C. till clear solution obtains. Added 75 mg charcoal and stirred for 10 min. Filtered the RM on hyflo bed and washed with 0.5 ml acetone. Cooled the filtrate to RT then to 0-5° C. and added 25 ml ether dropwise in the above suspension. Stirred the suspension for 1 H. Filtered and air-dried under 9 mbar vacuum to get 1.19 gm ERL free base purified (Yield=79.4%, Purity=99.36%).

### EXAMPLE 22

### Preparation of Pure Erlotinib Free Base

Into a reaction vessel charged Erlotinib free base crude (1 gm, 2.54 mmol) and 10 ml solution of Chloroform: methanol (9:1) in a reaction vessel and with continuous stirring to make it clear solution. Added 40 ml toluene dropwise and stirred the suspension for 1 H. Filtered and dried under 9 mbar vacuum at 50° C. to get 634 mg ERL free base purified (Yield=63.4%, Purity=99.53%).

### EXAMPLE 23

### Preparation of Pure Erlotinib Free Base

Into a reaction vessel charged Erlotinib free base crude (500 mg, 1.27 mmol) and 6 ml ethyl acetate in a reaction vessel and heat to 75-80° C. till clear solution obtains. Added 25 mg charcoal and stirred for 15 min. Filtered the RM on hyflo bed and washed with 1 ml ethyl acetate. Cooled the filtrate to RT then to 0-5° C. and stirred for 30 min. Filtered and air dried under 9 mbar vacuum for 30 min to get 420 mg ERL free base purified (Yield=84.80%, Purity=99.36%).

### EXAMPLE 24

### Preparation of Pure Erlotinib Free Base

Into a reaction vessel charged Erlotinib free base crude (200 mg, 0.51 mmol) and 2 ml butan-2-one in a reaction vessel and heat to 60-65° C. till clear solution obtains. Added 10 mg charcoal and stirred for 15 min. Filtered the RM on hyflo bed and washed with 0.5 ml butan-2-one. Cooled the filtrate to RT then to 0-5° C. and stirred for 1 H. Filtered and air dried under 9 mbar vacuum for 30 min to get 102 mg ERL free base purified (Yield=51.0%, Purity=99.47%).

### EXAMPLE 25

### Preparation of Pure Erlotinib Free Base

Into a reaction vessel charged Erlotinib free base crude (200 gm, 0.64 mmol) and 2 ml water in a reaction vessel and heat to 60-65° C. Added 5.5 ml methanol dropwise to make it clear solution. Stirred the clear solution for 30 min at 60-65° C. and cooled to RT. Stirred the suspension for 30 min. Filtered and air dried under 9 mbar vacuum for 30 min to get 130 mg ERL free base purified (Yield=65.0%, Purity=99.75%).

### EXAMPLE 26

### Preparation of Pure Erlotinib Free Base

Into a reaction vessel charged Erlotinib free base crude (1.5 gm, 3.81 mmol) and 60 ml mixture of methanol (38 ml) and water (22 ml) in a reaction vessel and heat to 65-70° C. Stirred for 1 H to make it clear solution. Cooled the clear solution to RT and then to 0-5° C. Stirred the suspension for 1 H at 0-5° C. Filtered and dried under 9 mbar vacuum at 60° C. for 4 H to get 1.12 gm ERL free base purified (Yield=74.86%, Purity=99.80%).

### EXAMPLE 27

### Preparation of Stable Polymorphic Form of Erlotinib Hydrochloride

Into a reaction vessel, added ERL-3 (1.0 gm, 2.54 mmol) and 30 ml Methyl isobutyl ketone under stirring. Applied heating to 60° C. to make it clear solution. Added 3.4 ml ethyl acetate HCl dropwise in the clear solution and stirred for 10 min. Cooled the suspension to RT and stirred for 1 H. Filtered, dried at 50° C., under 2 mbar vacuum for 4 H to get 984 mg stable polymorphic form of Erlotinib hydrochloride (Yield=90.3%, Purity=96.14%, Assay=98.26%, Melting Point=226±3° C.; DSC Endotherm peak first at 212.66 and second at 226.41° C.; XRD having peaks at PXRD peaks at 20 of 5.5, 18.8, 22.7, 24.6 and 26.1±0.2 degree and pattern substantially as in FIG. 1).

### EXAMPLE 28

### Preparation of Stable Polymorphic Form of Erlotinib Hydrochloride

Into a reaction vessel, added ERL-3 (1.0 gm, 2.54 mmol) and 40 ml isopropyl acetate under stirring. Applied heating to 60° C. to make a clear solution. Added 3.4 ml of ethyl acetate. HCl dropwise in the clear solution and stirred the RM for 2 H. Cooled the suspension to RT and stirred for 1 H. Filtered, dried at 50° C., under 2 mbar vacuum for 4 hours to get 957 mg stable polymorphic form of Erlotinib hydrochloride (Yield=87.8%, Purity=96.37%, Assay=98.36%; DSC Endotherm peak first at 210.74 and second at 226.40° C.).

### EXAMPLE 29

### Preparation of Stable Polymorphic Form of Erlotinib Hydrochloride

Into a reaction vessel, added ERL-3 (500 mg, 1.27 mmol) and 5 ml chloroform under stirring. Stirred the RM at room temperature for 30 min. to make it clear solution. Added 10 ml ether and 0.12 ml conc. HCl dropwise and stirred the suspension for 2 H. Filtered, dried at 60° C., under 2 mbar vacuum for 4 H to get 0.514 gm stable polymorphic form of Erlotinib hydrochloride (Yield=94.2%, Purity=99.36%, Assay=99.31, Melting Point=229±3° C.; DSC Endotherm peak first at 214.76 and second at 229.96° C.; XRD as in FIG. 1).

### EXAMPLE 30

### Preparation of Stable Polymorphic Form of Erlotinib Hydrochloride

Into a reaction vessel, added ERL-3 (500 mg, 1.27 mmol) and 9 ml chloroform under stirring. Stirred the RM at room temperature for 30 min. to make it clear solution. Added 18 ml ether and 0.13 ml conc. HCl dropwise and stirred the suspension for 4 H. Filtered, dried at 85° C., under 2 mbar vacuum for 4 H to get 495 gm stable polymorphic form Of Erlotinib hydrochloride (Yield=90.7%, Purity=99.54%, Assay=98.42%, DSC Endotherm peak first at 216.61 and second at 233.48° C.; XRD as in FIG. 1).

### EXAMPLE 31

### Preparation of Stable Polymorphic Form of Erlotinib Hydrochloride

Into a reaction vessel, added ERL-3 (1.0 gm, 2.54 mmol) and 12 ml chloroform under stirring. Stirred the RM at room temperature for 30 min. to make it clear solution. Clarified the solution by filtration. Added 0.468 ml conc. HCl and 15 ml ether dropwise and stirred the suspension for 4 H. Filtered, dried at 85° C., under 2 mbar vacuum for 4 H to get 982 mg stable polymorphic form of Erlotinib hydrochloride (Yield=89.8%, Purity=99.37%, Assay=99.65%; XRD as in FIG. 1).

### EXAMPLE 32

### Preparation of Stable Polymorphic Form of Erlotinib Hydrochloride

Into a reaction vessel, added ERL-3 (1.0 gm, 2.54 mmol) and 35 ml acetonitrile under stirring. Stirred the RM at room temperature for 30 min. to make it clear solution. Clarified the solution by filtration. Added 0.3 ml conc. HCl and 5 ml acetonitrile dropwise in the filtrate and stirred the suspension for 4 H at room temperature. Filtered, dried at 85° C., under 2 mbar vacuum for 4 H to get 1.037 gm stable polymorphic form of Erlotinib hydrochloride (Yield=94.9%, Purity=99.01%, Assay=98.80%; XRD as in FIG. 1).

### EXAMPLE 33

### Preparation of Stable Polymorphic Form of Erlotinib Hydrochloride

Into a reaction vessel, added ERL-3 (1.0 gm, 2.54 mmol) and 35 ml acetone under stirring. Stirred the RM at room temperature for 30 min. to make it clear solution. Clarified the solution by filtration. Added 0.3 ml conc. HCl and 15 ml acetone dropwise in the filtrate and stirred the suspension for 4 H at room temperature. Filtered, dried at 85° C., under 2 mbar vacuum for 4 H to get 1.041 gm stable polymorphic form of Erlotinib hydrochloride (Yield=92.8%, Purity=98.98%, Assay=98.99%; XRD as in FIG. 1).

### EXAMPLE 34

### Preparation of Stable Polymorphic Form of Erlotinib Hydrochloride

Into a reaction vessel, added ERL-3 (700 mg, 1.78 mmol) and 8.4 ml chloroform under stirring. Stirred the RM at room temperature for 30 min. to make it clear solution. Added 14 mg charcoal and applied heating to 45-50° C. for 15 min. Filtered the RM on hyflo and washed with 1 ml chloroform. Added 1.3 ml ethereal HCl and 10 ml ether dropwise in the filtrate and stirred the suspension for 4 H at room temperature. Filtered, dried at 85° C., under 2 mbar vacuum for 4 H to get 668 mg stable polymorphic form of Erlotinib hydrochloride (Yield=87.6%, Purity=98.59%, Assay=98.40%; XRD as in FIG. 1).

### EXAMPLE 35

### Preparation of Stable Polymorphic Form of Erlotinib Hydrochloride

Into a reaction vessel, added ERL-3 (1.0 gm, 2.54 mmol) and 40 ml chloroform under stirring. Stirred the RM at room temperature for 30 min. to make it clear solution. Added 50 mg charcoal and applied heating to 45-50° C. for 30 min. Filtered the RM on hyflo and washed with 2 ml chloroform. Added 2.75 ml ethereal HCl and 10 ml ether dropwise in the filtrate and stirred the suspension for 4 H at room temperature. Filtered, dried at 85° C., under 2 mbar vacuum for 4 H to get 1.02 gm stable polymorphic form of Erlotinib hydrochloride (Yield=93.3%, Purity=99.83%, Assay=99.86% Melting Point=231±3° C.; DSC Endotherm peak first at 214.68 and second at 230.30° C.; XRD as in FIG. 1).

1. A stable polymorph of Erlotinib Hydrochloride characterized by Differential Scanning Calorimetry (DSC) that corresponds to FIG. 2 having one sharp endotherm peak in the range between 214-224° C. and another endotherm sharp peak in the range between 229±−239° C.

2. Erlotinib Hydrochloride as claimed in claim 1, further characterized by melting point between 229±3° C.

3. Erlotinib Hydrochloride as claimed in claim 1 characterized by PXRD peaks at 2Θ of 5.5, 18.8, 22.7, 24.6 and 26.1±0.2 degrees.

4. Erlotinib Hydrochloride as claimed in claim 1, exhibiting PXRD pattern that corresponds to FIG. 1.

5. Erlotinib Hydrochloride as claimed in claim 1, which is further characterized by FT-IR that corresponds to FIG. 3.

6. Erlotinib Hydrochloride as claimed in claim 1 which is further characterized by Microscopic pattern that corresponds to FIG. 4.

7. A process for the preparation of stable polymorph of Erlotinib Hydrochloride, comprising;
a) chlorination of compound of formula IV with suitable chlorinating reagent in the presence of suitable solvent and optionally suitable anti-solvent to get compound of formula III 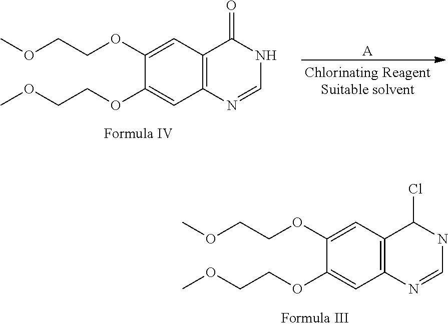
b) reacting compound of formula III with 3-ethynyl benzenaminc in the presence of suitable base and suitable solvent to get compound of formula II, 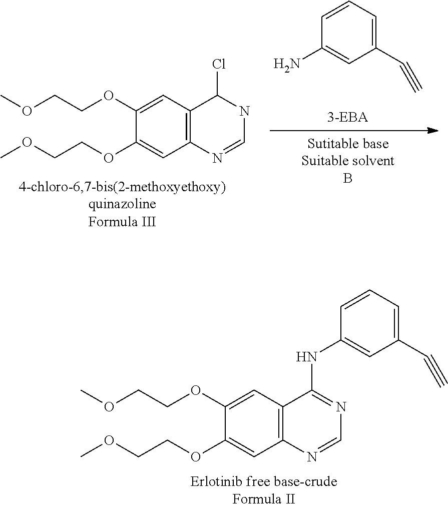
c) purification of compound of formula II with suitable solvent and optionally suitable anti-solvent to get the pure compound of formula II 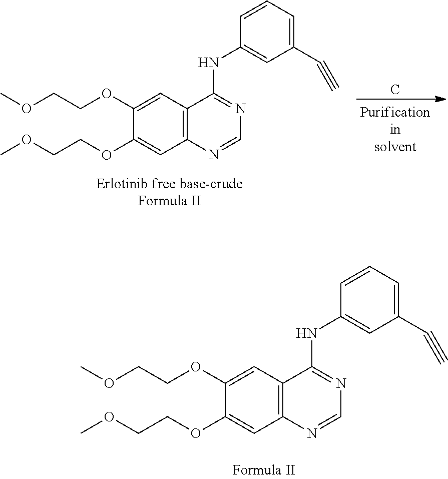
d) reacting the compound of formula II with HCl to give Erlotinib hydrochloride of formula I

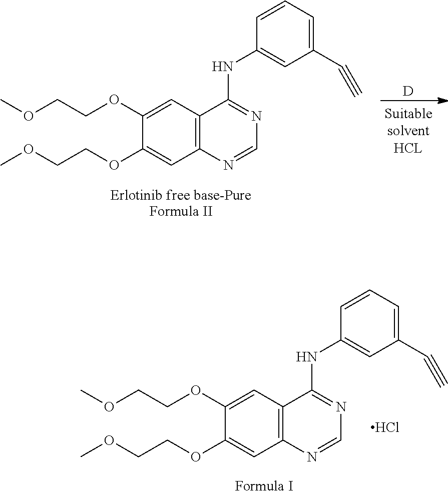

8. A process as claimed in claim 7, wherein in step (a), said chlorinating reagent is selected from thionyl chloride, phosphoryl chloride, phosphorous pentachloride, oxalyl chloride, methane sulphonyl chloride, benzene sulphonyl chloride, aniline sulphonyl chloride or mixture thereof; suitable solvent is selected from toluene, dichloromethane, chloroform, acetonitrile, cyclohexane or mixture thereof; suitable anti-solvent is selected from isopropyl alcohol, heptane, hexane, toluene or mixture thereof and suitable base is selected from pyridine, triethyl amine, n-methyl morpholine, collidine, N, N dimethyl aniline or a mixture thereof.

9. A process as claimed in claim 7 wherein step (b), said suitable solvent is selected from C₁ to C₆ alcohols, dichloromethane, chloroform, DMF, N-methylpyrrolidin-2-one, acetonitrile, tetrahydrofuran, 1,4-dioxane or suitable mixtures thereof; step C, suitable solvent is selected from chloroform, methanol, butan-2-one, ethyl acetate, water, acetone or mixture thereof; and step C, suitable anti-solvent is selected from toluene, hexane, water, ether or mixture thereof.

10. A process as claimed in claim 7, wherein step (d), said suitable solvent is selected from chloroform, methyl isobutyl ketone, isopropyl acetate, acetone, acetonitrile, dichloromethane, dioxane, ether or mixture thereof.

11. A process as claimed in claim 7, wherein step (d), said HCl is added to an organic solvent selected from suitable ethers, alcohols or an aqueous solution.

12. A process for the purification of crude Erlotinib base into pure Erlotinib base comprises, addition of suitable solvent and optionally suitable anti-solvent into crude Erlotinib base to obtain pure Erlotinib base.

13. The process for the purification as claimed in claim 12, wherein suitable solvent is selected from chloroform, methanol, butan-2-one, ethyl acetate, water, acetone or mixture thereof and suitable anti-solvent is selected from toluene, hexane, water, ether or mixture thereof.

14. The pure Erlotinib base prepared by the process as claimed in claim 12, having purity more than 99.80% by HPLC.

15. The stable polymorph form of Erlotinib hydrochloride as prepared by the process of claim 7, having purity more than 99.8% by HPLC.
